(19) United States
(12) Patent Application Publication
(10) Pub. No.: US 2013/0133670 A1
(43) Pub. Date: May 30, 2013

(54) SURGICAL DRAPE

(71) Applicant: Klaus JUNK, Marburg (DE)

(72) Inventor: Klaus JUNK, Marburg (DE)

(21) Appl. No.: 13/744,749

(22) Filed: Jan. 18, 2013

Related U.S. Application Data
(63) Continuation of application No. 10/562,215, filed on Mar. 9, 2007, now abandoned.

(30) Foreign Application Priority Data
Jun. 26, 2003 (DE) 103 28 907.0

(51) Int. Cl. A61B 19/08

(52) U.S. Cl. CPC A61B 19/088
USPC 128/854

(57) ABSTRACT

The invention concerns a novel surgical drape with a first fenestration for an incise film and a collection pouch related to this film for surgical residues according to the state of the art. The drape features at least one further fenestration for the reception of a cover for a reference device protruding from the level of the surface of the drape, which is identifiable for a 2- or 3-dimensional measurement system.

### DESCRIPTION AND TECHNICAL STATE OF THE ART

The invention concerns a surgical drape, in particular for surgery of the head.

Surgical drapes serve to keep the area of the surgical incision site as clean and sterile as possible. Most of the drapes feature in their main sheet, for that purpose, apertures formed according to the incision site, so called “fenestrations”, or these are correspondingly cut. Within the fenestration, so-called “incise drapes or foils” are then adhered. Such drapes can be obtained from several producers already assembled with pre-cut incise films inserted in standardized dimensions.

After the skin of the patient has been cleaned and disinfected in the incision site, the incise film is usually adhered to the skin in the area of the surgical incision site and allows—since the films are usually transparent—direct intervisibility with the border area of the surgical incision site as well. For fixation, the incise films feature special bonding surfaces, which are usually covered by removable protection films.

In the state of art, several such drapes, including “incise drapes or films” are known.

For example, DE 26 02 562 A1 (based on U.S. application Ser. No. 545,288) and DE 26 02 563 A1 (based on US application number 545 289) (from applicant Johnson & Johnson and a priority date of Jan. 29, 1975) show, respectively, a surgical drape consisting of a main sheet, comprised of a flexible, adaptable material, a collar fastened to the upper area of the main sheet with hands-receiving pockets, and an adhesive strip in order to attach the drape in a removable manner to the body (in particular the head) of the patient. Both of the aforementioned drapes feature several complex fold lines. Both serve the purpose of shortening the preparation time of an operation and ensuring sterile coverage.

U.S. Pat. No. 3,791,382, quoted in DE 29 15 627 C2 (Johnson & Johnson, application date Apr. 18, 1979), features pouches for receiving fluids which typically occur during a cranial operation which are located on the surface of such a drape in the area which hangs under the operating table. DE 29 15 627 C2 offers an improvement in the form of specially formed cloths which should ensure that fluids which possibly occur are in fact securely lead to such a pouch.

A newer document (U.S. Pat. No. 6,129,085, as a divisional application of Ser. No. 08/705,689 applied on Aug. 30, 1996, U.S. Pat. No. 5,778,889, Kimberly-Clark) likewise proposes a surgical drape (“craniotomy drape” corresponds to “cranial opening drape”, in particular for an operation on the cranium. The drape presented in that patent should allow the anesthetist eye-contact with the face of the patient at all times throughout the operation; furthermore, the drape should not trail on the floor of the operating room; finally, the drape should feature easily adjustable and weight-bearing clamps for the attachment of instruments, tubes, etc. During the execution of all tasks the sterility of the room in the area of the patient should remain maintained. The aforementioned U.S. Pat. No. 5,778,889 proposes, to solve this task, a craniotomy drape with a run-off collection pouch below the head area of the patient to be operated on whereby the drape features on the sides of the main sheet a “gusset” or a “corner drape” as well as lateral, flat and transparent parts in one embodiment. Both parts (the gusset and the transparent lateral parts) are measured in such a way that they prevent the drape from reaching the floor (depending on the height of the operating table). The drape features further in another embodiment holders for receiving instruments, or cables and tubes respectively.

DE 195 10 020 A1 (General Electric, application date Mar. 29, 1994 from U.S. Pat. No. 2,195,79) proposes a drape which contains a coil between two layers to enable MRI imaging to be performed during operations.

Publications which deal directly or almost exclusively with “incise sheets” or films, etc. are, e.g.:

CA 2 314 962 (application date Jan. 8, 1999) proposes a special point- and ring-shaped arrangement of the adhesive surfaces.

U.S. Pat. No. 5,979,450 (applicant 3M, application date Apr. 9, 1998) provides for an incise or pre-cut film or drape which is reinforced by the use of covering sheets and, if necessary, additional tensioning strips (“liners”) in order to be able to fasten the incise film in a wrinkle-free state on the body of the patient.

EP 0 902 660 B1 (applicant 3M, priority date as indicated in the U.S. priority-certificates Ser. No. 08/648,903 and Ser. No. 08/724,744, the earliest on May 16, 1996) proposes, for the same purpose, an incise film without covering sheets (liners) which can be unrolled.

EP 0 568 401 B1 (applicant Laboratoire Hydrex (SA), FR, application date with priority of FR 92 052 50, Apr. 29, 1992) proposes, for the same purpose of “ wrinkle-free fastening capacity”, a (such as EP 0 902 660 B1) three-layer incise film as well, however one which features a covering sheet for the reinforcement of the transparent lamination on the other side and in addition to that a perforation which extends through all three layers.

Due to further development within surgery, the exact location of the surgical incision site is measured 2- or 3-dimensionally in the time before the incision (e.g. by MRI, MRT, x-ray or ultrasound) in order to localize the area as precisely as possible and with that, e.g. to protect healthy tissue as much as possible.

After this “determination”, the surgical incision occurs in the operating room, whereby other or further 3-dimensional measurement systems, e.g. so-called navigation systems, which work, e.g., with two infrared emitters and receivers, are used to retain the knowledge of the precise location of the surgical incision site. In total, a modification of the bench marks for coordinates is necessary in this for the surgical incision site. For the implementation of this modification, a means of reference somehow shaped geometrically in a suitable manner identifiable from the second measurement system (called navigation system in the following) is fixed mostly in the area of the surgical incision at the operating table or another location in the operating room with a permanent reference to the surgical incision. The second measurement system (navigation system) for this does not necessarily have to be different from the first measurement system.

Thus in the meantime, 2- or 3-dimensional navigation systems, for example, are often used with the corresponding number of, e.g. infrared emitters and receivers which comprise a means of reference, e.g. in the form of a three-armed star (called a navigation basis unit).

By moving the pictures of the incision site produced by the first and second measurement systems “one above the other”, e.g. on a suitable monitor, the modification of the bench marks for coordinates is then performed. In order not to lose the reference of the position of the incision site during the operation to the position of the incision site during the first, usually more precise measurement, it is therefore necessary for the navigation system, during the operation as well, to interact more or less permanently with the means of reference, so that the means of reference should be fixed, if possible, outside of the area of the surgeons' movements as possible. On the other hand, the means of reference are to be fixed advantageously near the incision site.

As a compromise, the necessity results then, apart from the incision site, to also cover the means of reference in a as sterile as possible manner but to remain identifiable for the navigation system. This task is made more difficult in the cases where the means of reference—for reasons of work ergonomics for the operating team—is fixed at a level which is outside of the work area of the operating team and with that usually above the incision site.

In the state of the art, in particular from the aforementioned publications, there is no feasible solution known for that. In the state of the art there are only such surgical drapes known which feature, apart from a fenestration for an incise film, another—attached to one side of the drape—fluid-impervious pouch in the area between the upper border of the incise film and the upper end of the drape. Furthermore known drapes usually still feature a means of tensioning on the lateral borders of the incise film and in the area of entry of the fluid-impervious pouch. Usually these means of tensioning, e.g. in the form of metal strips, serve as a guide for fluids or surgical residues, so that all of these come to be in the pouch.

### Tasks of the Invention

It is, therefore, the task of the invention to provide a novel surgical drape which apart from enabling sterile cover of the surgical incision site, also enables sterile cover for means of reference shaped somehow for known measurement systems, in particular measurement systems using infrared radiation.

### Practical Embodiments

This task is solved by the subject matter of patent claim 1.

This problem is solved with a surgical drape with a first fenestration for an incise film with or without a collection pouch related to that film for surgical residues according to the state of the art, characterized by the fact that the drape features at least one further fenestration for the reception of a means of cover for a means of reference protruding from the level of the drape's surface which is identifiable for a 2- or 3-dimensional measurement system.

With that, the advantage of also sterilely covering a means of reference next to the patient, something that is common for modern—equipped with 2- or 3-dimensional measurement devices—operating rooms, is achieved without limiting the mobility of the means of reference or having to break the border between the sterile and non-sterile operating area during the operation.

The entire novel drape can, therefore, be sterilized in one go. With that a significant gain in time results for the preparation of the operation as well, which can, for example, be dedicated to the patient.

Further advantages result from the subject matter of the respective subclaims, or the production methods claimed.

Further details and advantageous realizations of the invention result from the practical embodiments described in the following and depicted in the figures, as well as from the subclaims below. It shows:

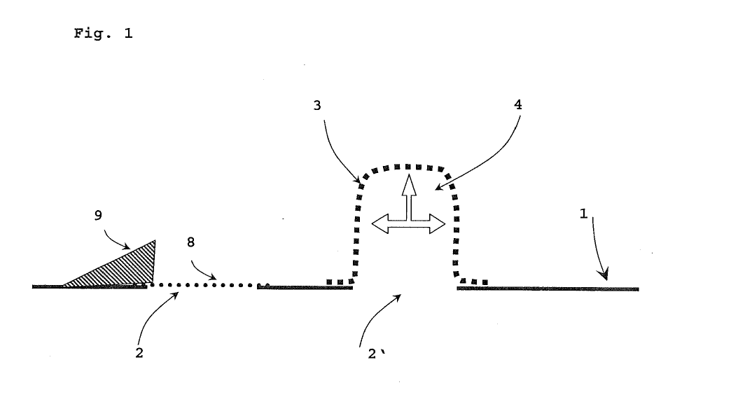

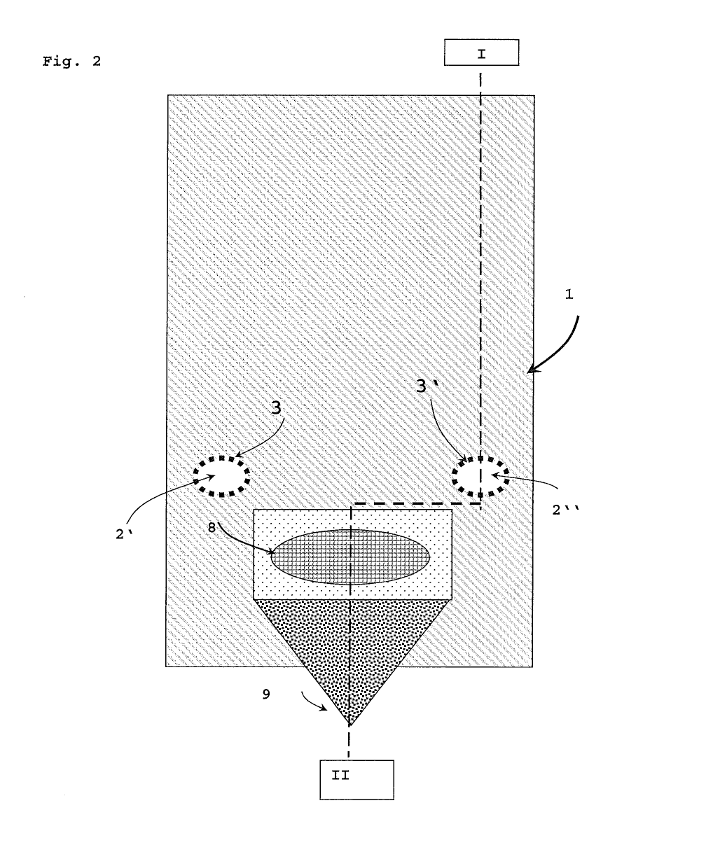

FIG. 1 a cross-section of the drape according to the present invention along the line I-II from FIG. 2

FIG. 2 a view from above of the drape according to the present invention with two means of cover

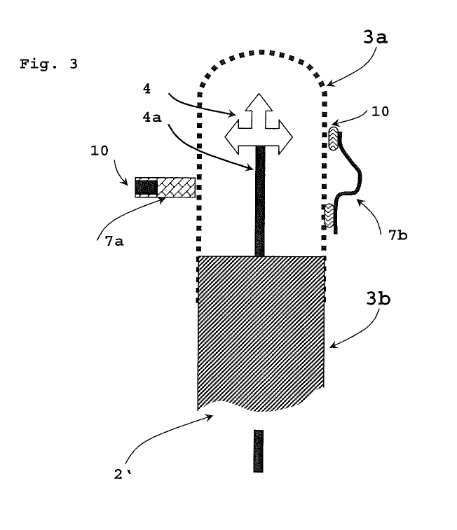

FIG. 3 a cross-section of a means of cover according to the present invention

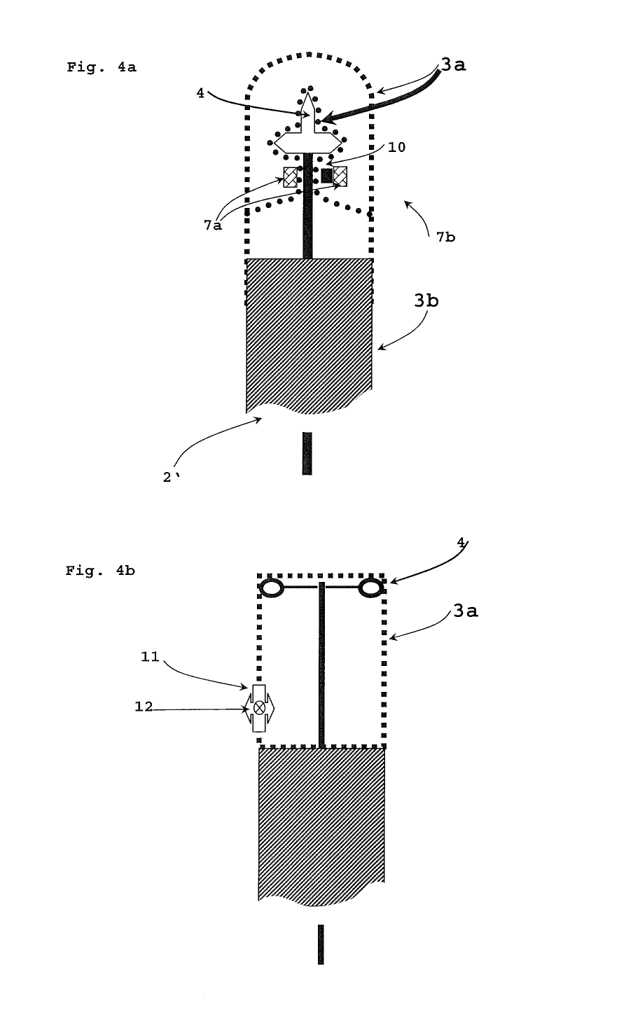

FIG. 4a a cross-section of an embodiment of the means of cover according to the present invention

FIG. 4b a cross-section of a further embodiment of the means of cover according to the present invention

FIG. 1 shows an embodiment of the means of cover according to the present invention in a cross-section of the line I-II from FIG. 2. In this, the means of reference 4 covered by the means of cover 3, which is bound with the drape 1 at the perimeter of a second fenestration (2′), is depicted. In the left part of the figure, the first fenestration 2 for the incise film 8 is depicted. Above that, in the direction of the border of the drape 1 is depicted the fluid collection pouch 9 related to the incise film.

FIG. 2 shows a view from above of the drape according to the present invention, in this embodiment with two means of cover, which measure approx. 25 cm in diameter respectively and are arranged approx. 50 cm away from the center of the incise film. Through this, swinging the means of reference to the other side of the operating table is easily performable even during an operation.

Likewise the incise film 8 is depicted as well as the fluid collection pouch 9.

FIG. 3 shows a cross-section of a further advantageous embodiment of the means of cover 3, depicted with the means of reference 3 in the form of a three-armed indicator, which is fastened to a holder 4a. A non-transparent part 3b is bound to the upper part 3a which is transparent for the known measurement systems. This can be produced, e.g. from stiffer and/or tougher material than the upper part, so that the entire means of cover 3 stands more or less on its own, without support, on a holder.

On the left side, a means of reduction 7a is depicted with which the perimeter of the means of cover can be reduced vertically to the longitudinal axis. In this embodiment, i.e. only as an example, a strip with an adhesive element is provided for this.

For shortening the means of cover, a means of shortening 7b, depicted on the right side of the figure, is provided, which is likewise equipped with adhesive elements 10. Thus, the means of cover can be adjusted to the means of reference and its position in a simple manner (e.g. variable by a moveable holder), avoiding unnecessary creases to distort the radiation coming from the measurement system by the means of cover or the reflected radiation from the means of reference (e.g. by scattering, etc.).

FIG. 4a shows the means of cover 3 in a form (round points) which has been adjusted to the form of the means of reference by the means of reduction 7a.

This adjustment can also occur by pre-shaping of the means of cover, whereto the upper end of the means of cover features corresponding shapes.

FIG. 4b shows a further advantageous embodiment of the means of cover 3, whereby this time a flat end ensures a good fit with the means of reference formed in this case by a three-armed star with balls as reference points.

A particularly preferred embodiment is given in that the means of cover is designed with elastic or plastic transparent materials, whereby the means of cover features simultaneously the capacity to be inflated. Therewith, any formation of creases, etc. is prevented. For this purpose, the bottom end on the means of cover is manufactured to be closed and features a valve on the surface and, if necessary, means of pumping.

It is immediately obvious that the means of cover must not necessarily be manufactured elongated or in the form of a cylinder with one or two closed ends. Every other form can likewise contribute to the desired sterile coverage of a means of reference.

Hereby, means of reference is understood to be any means which, through the known measurement or navigation systems in the running of a hospital, is suitable to mark a point in a 2- or 3-dimensional space or to relate with that a coordinate point of reference.

### LIST OF REFERENCE NUMERALS

Drape 1
Fenestration 2, 2′, 2″, 2′″
Means of cover 3
Means of cover, transparent area 3a
Means of cover, non-transparent area 3b
Means of reference 4
Holder for means of reference 4b
Surgical incision site 5
Measurement system, navigation system 6
Means of reduction 7a
Means of shortening 7b
Incise film 8
Fluid collection pouch 9
Adhesive element 10
Valve 11
Means of pumping 12

1. Surgical drape for covering operating sites on a body of a patient comprising:
a top and bottom surface;
a first fenestration in the drape, the first fenestration covered by an incise film aligned with the top surface of the drape;
a second fenestration in the drape spaced from the first fenestration and defining a perimeter;
a means of cover firmly bound along the entire perimeter of the second fenestration, the means of cover having an elongated form which extends upwardly from the top surface of the surgical drape and terminates in a closed upper end, the means of cover including a transparent portion, the transparent portion spaced from the top surface of the surgical drape to permit transmission of radiation by a measurement system to be used in conjunction with a means of reference.

2. Drape according to claim 1, characterized by the fact that the means of cover is made of a flexible material.

3. Drape according to claim 1, characterized by the fact that the means of cover features along the surface between the drape and the upper end at least one means of reduction, for the reduction of the perimeter of the means of cover.

4. Drape according to claim 1, characterized by the fact that the means of cover features at least one means of reduction along the surface between the drape and the closed upper end for the reduction of the length of the means of cover, so that the closed upper end can also be stretched firmly and smoothly over the means of reference, whereby creases or other distortions of the radiation of the measurement system falling on the means of reference or reflected therefrom are avoided.

5. Drape according to claim 5, characterized by the fact that the means of reduction are realized in the form of removable adhesive strips or simple cords.

6. Drape according to claim 1, characterized by the fact that the means of cover features pre-shaped moldings on an upper, drape-opposing end for the reception of shapes of the means of reference.

7. Drape according to claim 1, characterized by the fact that the means of cover is realized to be able to be sterilized.

8. Drape according to claim 1, characterized by the fact that the drape is realized attached firmly to the means of cover along the fenestration by the technology of ultrasonic welding, adhesion or heat welding.

9. Drape according to claim 1, characterized by the fact that the means of cover is realized with an elastic or plastic material and in the area of the coverage of the means of reference is inflatable so that formation of creases that would distort the radiation directed to or reflected from the means of reference are avoided.

10. Drape according to claim 1, further comprising at least two second fenestrations and at least two means of cover, one means of cover for each second fenestration.

11. Drape according to claim 1, wherein the means of cover, measured from the middle point of the fenestration surface at the foot of the means of cover, feature a distance of 10 to 100 cm from the center of the incise film.

12. Drape according to patent claim 10, characterized by the fact that one of the at least two means of cover, respectively, are arranged at a distance of approx. 40 cm left and right in a perpendicular distance from the center of the incise film, so that during an operation, swinging the means of reference from one side to the other can be easily undertaken.

13. Drape of claim 2, wherein the flexible material is a polymer.

14. Drape of claim 7, wherein the means of cover can be sterilized with gamma radiation or hot steam.

15. Drape of claim 1, wherein the means of cover is firmly bound to the top surface of the drape.

16. Drape of claim 10, wherein the at least two means of cover have a minimum outside diameter of 10-50 cm.

17. Drape according to claim 11, wherein the distance is 50 cm from the center of the incise film.

18. Drape according to claim 1, wherein the means of cover is a different material than the material of the incise film covering the first fenestration.

19. Drape according to claim 1, wherein a bottom portion of the cover comprises a material that is stiffer than an upper end portion of the cover.
